(19) United States
(12) Patent Application Publication
(10) Pub. No.: US 2023/0338054 A1
(43) Pub. Date: Oct. 26, 2023

(54) MEDICAL DEVICE

(71) Applicant: TERUMO KABUSHIKI KAISHA, Tokyo (JP)

(72) Inventor: Yuichi TADA, Santa Clara, CA (US)

(73) Assignee: TERUMO KABUSHIKI KAISHA, Tokyo (JP)

(21) Appl. No.: 18/347,672

(22) Filed: Jul. 6, 2023

Related U.S. Application Data
(63) Continuation of application No. PCT/JP2021/045091, filed on Dec. 8, 2021.

(30) Foreign Application Priority Data
Jan. 11, 2021 (JP) 2021-002497

(51) Int. Cl. A61B 17/3207; A61M 1/00

(52) U.S. Cl. CPC A61B 17/3207; A61M 1/74; A61M 1/80; A61M 2205/3337

(57) ABSTRACT

A medical device exhibits an improved ability to discriminate the nature of a discharging flow. The medical device that discharges an object from a biological body lumen includes a shaft that extends from a proximal side toward a distal side and includes a discharge lumen through which the object can pass to the proximal side, a discharge passage in communication with a proximal portion of the discharge lumen, and a mixing unit capable of supplying fluid to the discharge passage.

### CROSS-REFERENCES TO RELATED APPLICATIONS

This application is a continuation of International Patent Application No. PCT/JP2021/045091 filed Dec. 8, 2021, which claims priority to Japanese Patent Application No. 2021-002497 filed on Jan. 11, 2021, the entire content of both of which is incorporated herein by reference.

### TECHNOLOGICAL FIELD

The present invention generally relates to a medical device for discharging an object in a biological body lumen.

### BACKGROUND DISCUSSION

In recent years, a medical device that discharges (removes) a thrombus or the like from a biological body lumen such as a blood vessel to the outside of the body has been used. For example, an atherectomy device is used to cut and discharge a stenosis area caused by a plaque, a thrombus, or the like in a blood vessel. The atherectomy device has a function of collecting debris generated during the cutting into the device using a suction means provided in a drive unit.

### SUMMARY

The object such as the debris collected from the biological body lumen into the device may stay without smoothly flowing in the device. In this case, presence or absence of a flow in the device needs to be determined. However, it is difficult to determine the presence or absence of the flow only by visual appearance.

For example, Japanese Patent No. 4540227 (JP 4540227 B1) discloses a method of determining stoppage of a flow of a fluid by measuring a time differential pressure change of the fluid. However, this method requires the measurement of pressure, and it is difficult to recognize the presence or absence of the flow in the device only by the visual appearance.

The medical device is capable of improving discriminability of a discharging flow.

A medical device for achieving a result like that described above is a medical device that discharges an object from a biological body lumen, and includes a shaft that extends from a proximal side toward a distal side and includes a discharge lumen through which the object can pass to the proximal side, a discharge passage that communicates with a proximal portion of the discharge lumen, and a mixing unit capable of supplying, to the discharge passage, fluid or a moving body movable in the discharge passage.

The medical device configured as described above is capable of mixing, using the mixing unit, the fluid or the moving body into a flow toward the proximal side through the discharge lumen. Accordingly, the medical device is enabled to improve discriminability of presence or absence of a discharging flow by creating appearance contrast in the flow.

According to another aspect, a medical device that discharges an object from a biological body lumen comprises: a shaft that includes a rotatable part having a proximal portion and a distal portion, with the shaft including a discharge lumen having an open distal end, the discharge lumen extending from the open distal end to a proximal portion of the discharge lumen; a cutting unit fixed to the distal portion of the rotatable part; and a motor connected to the rotatable part to rotate the rotatable part and the cutting part so that during use of the medical device when the motor is operated, the cutting unit is rotated to cut the object. A suction producer is in fluid communication with the discharge lumen so that operation of the suction producer during use of the medical device produces suction in the discharge lumen that causes the object cut by the cutting unit to enter the discharge lumen by way of the open distal end. A discharge passage communicates with the proximal portion of the discharge lumen, and a mixing unit supplies fluid or a moving body to the discharge passage during use of the medical device.

According to another aspect, a medical device that discharges an object from a biological body lumen comprises: a shaft that includes a discharge lumen opening to a distal portion of the shaft and extending to a proximal portion of the shaft; a discharge passage that communicates with the discharge lumen; and a mixing unit that supplies fluid or a moving body to the discharge passage during use of the medical device. The mixing unit and the discharge passage are enclosed within an enclosure of an operation unit, and a proximal portion of the shaft is also located in the enclosure of the operation unit. The mixing unit that is enclosed within the enclosure of the operation unit comprises a main flow path positioned intermediate opposite ends of the discharge passage, with the main flow path having an inflow portion and an outflow portion that are spaced apart from one another. The mixing unit that is enclosed within the enclosure of the operation unit also comprises a mixing flow path that intersects and is in fluid communication with the main flow path at a location along the main flow path that is intermediate the inflow portion and the outflow portion. The mixing unit that is enclosed within the enclosure of the operation unit also comprises a one-way valve allowing the fluid or the moving body to be introduced into the discharge passage by way of the main flow path.

### BRIEF DESCRIPTION OF THE DRAWINGS

FIG. 1 is a plan view illustrating a medical device according an embodiment.

FIG. 2 is a cross-sectional view illustrating a proximal portion of the medical device according to the embodiment.

FIG. 3 is a plan view illustrating a mixing unit of the medical device according to the embodiment.

FIG. 4 is a view illustrating a variation in which a part of an operation unit is illustrated in a cross-section and other parts are illustrated in a plane.

### DETAILED DESCRIPTION

Hereinafter, an embodiment of the medical device, representing an example of the new medical device disclosed here, will be described with reference to the drawings. The size and ratio of each member in the drawings may be exaggerated for explanatory convenience, and may be different from the actual size and ratio.

A medical device 10 according to the present embodiment is inserted into a blood vessel, and used for a procedure for destroying and discharging (removing) an object such as a thrombus, a plaque, an atheroma, or a calcified lesion in acute lower limb ischemia or deep vein thrombosis. In the present specification, a side of the device to be inserted into a blood vessel is referred to as a “distal side”, and a side to be operated is referred to as a “proximal side”. The object to be destroyed and discharged is not necessarily limited to the thrombus, the plaque, the atheroma, and the calcified lesion, and may be any object that may exist in a biological body lumen. In addition, a side toward which waste liquid to be discharged flows (side from upstream to downstream) is referred to as a “discharge side” in the present specification.

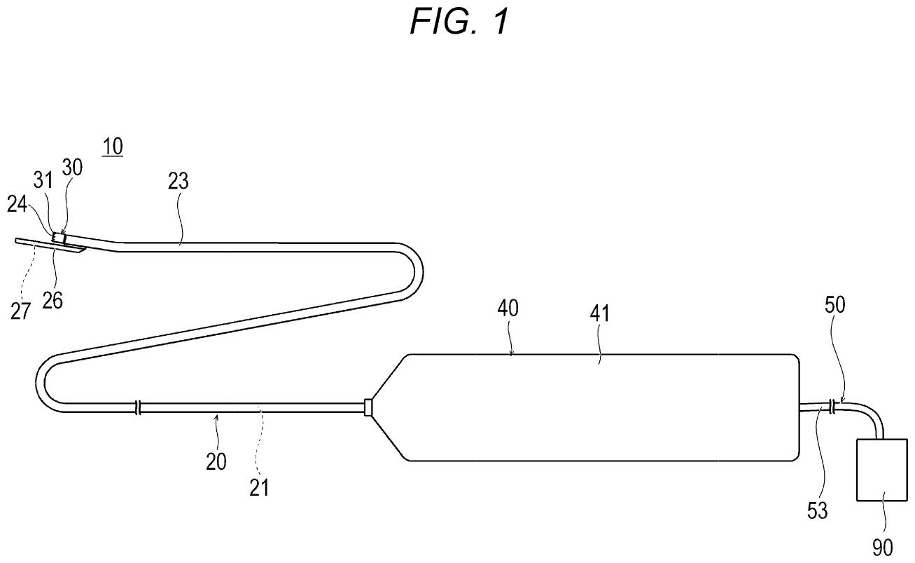

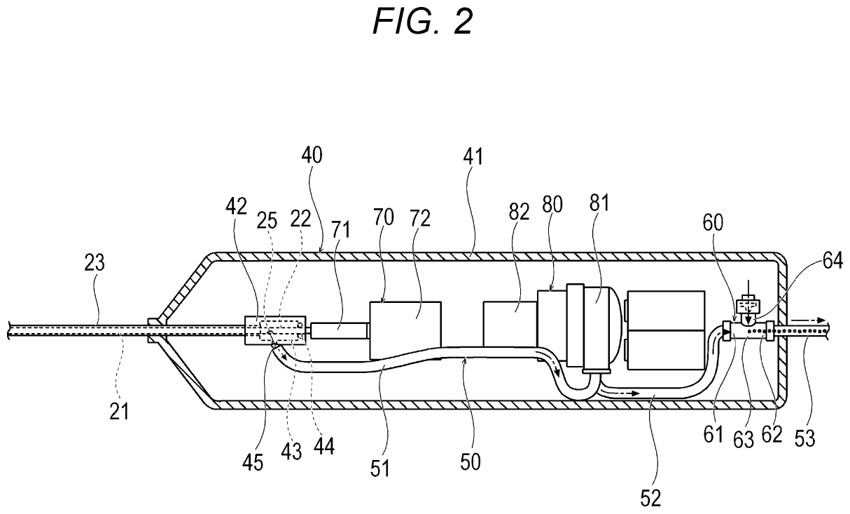

As illustrated in FIGS. 1 and 2, the medical device 10 includes a long shaft 20 having a discharge lumen 21 through which an object can pass to the proximal side, a cutting unit 30 coupled to the distal portion of the shaft 20, and an operation unit 40 to which the proximal portion of the shaft 20 is coupled. The medical device 10 further includes a discharge passage 50 in communication with the discharge lumen 21 provided in the shaft 20, a mixing unit 60 capable of supplying fluid to the discharge passage 50, a drive unit 70 that rotates a drive shaft 22 provided in the shaft 20, a suction unit 80 coupled to the discharge passage 50, and a drainage bag 90.

The shaft 20 includes the drive shaft 22 rotationally driven by the drive unit 70, an outer tube 23 that rotatably houses the drive shaft 22, and a distal tube 26 fixed to a side surface of a distal portion of the outer tube 23.

The drive shaft 22 is coupled to the cutting unit 30, and transmits torque to the cutting unit 30. The drive shaft 22 is flexible, and has a property of transmitting rotative power applied from the proximal side to the distal side. The drive shaft 22 is provided with the discharge lumen 21 for moving a cut object toward the proximal side. The drive shaft 22 penetrates or passes through the outer tube 23, and has a distal portion to which the cutting unit 30 is fixed. The drive shaft 22 has a proximal portion coupled to the drive unit 70. The drive shaft 22 has a distal end provided with a distal end opening 24 at which the discharge lumen 21 opens. The distal end opening 24 is an inlet through which debris, which is a suction target formed when being cut, enters. That is, debris resulting from operation of the cutting unit 30 is the target of suction produced by the suction unit 80 and this suction draws the debris into the distal end opening 24 that communicates with the discharge lumen 21. The proximal portion of the drive shaft 22 is provided with a proximal end opening 25. The proximal end opening 25 is an outlet through which the debris, which has entered the inside of the drive shaft 22 from the distal end opening 24, is discharged. The discharge lumen 21 is not necessarily disposed inside the drive shaft 22, and may be disposed between the outer tube 23 and the drive shaft 22 or in another tube provided inside the drive shaft 22.

The cutting unit 30 is a member that cuts an object such as a thrombus, a plaque, or a calcified lesion into a small size. Therefore, the term “cut” indicates the action of applying force to an object in contact to make the object smaller. A method for applying force during the cutting and a shape or a form of the cut object are not limited. The cutting unit 30 has strength capable of cutting the object mentioned above. The cutting unit 30 is fixed to the distal portion of the drive shaft 22. The cutting unit 30 is a cylinder protruding toward the distal side further than the drive shaft 22. The cutting unit 30 may be hollow and in communication with the discharge lumen 21. The cutting unit 30 has a distal end provided with a sharp blade 31. A shape of the blade 31 is not particularly limited. The cutting unit 30 may have a large number of fine abrasive grains instead of the blade 31.

The drive unit 70 rotates the drive shaft 22. The drive unit 70 includes a rotary drive shaft 71 and a first motor 72 that rotates the rotary drive shaft 71. The rotary drive shaft 71 is fixed to the proximal portion of the drive shaft 22. A rotation speed of the first motor 72 is not particularly limited, and is, for example, 5,000 to 200,000 rpm.

The suction unit 80 includes a pump 81 coupled to the discharge passage 50, and a second motor 82 that rotates the pump 81. The pump 81 is driven by the second motor 82 to apply negative pressure to the upstream side of the discharge passage 50. The pump 81 moves drainage sucked through the discharge lumen 21 and the discharge passage 50 to the downstream side, and discharges the drainage to the drainage bag 90. The pump 81 is, for example, a peristaltic pump. The peristaltic pump partially crushes or squeezes a tube by a plurality of rotating rollers, and the position of the crushed or squeezed portion moves, thereby moving fluid inside the tube. Since the fluid is not contained in the crushed position of the tube, the peristaltic pump is enabled to intermittently move the fluid. The peristaltic pump can reliably isolate the upstream side and the downstream side from each other by the crushed portion of the tube. Therefore, the peristaltic pump is capable of effectively avoiding a back-flow of the fluid mixed by the mixing unit 60 toward the upstream side, and is highly secure. A form of the pump 81 is not particularly limited. The pump 81 may be, for example, a diaphragm pump capable of reliably isolating the upstream side and the downstream side from each other. A flow velocity in the discharge passage 50 generated by the pump 81 is not particularly limited, and is, for example, 20 to 30 mm/min. A flow rate in the discharge passage 50 generated by the pump 81 is not particularly limited, and is, for example, 250 to 460 mm³/min.

The outer tube 23 is a flexible tube, and has a proximal portion fixed to the operation unit 40. The outer tube 23 has a distal portion positioned on the proximal side of the cutting unit 30. The distal tube 26 is a flexible tube, and is fixed to the outer periphery of the distal portion of the outer tube 23. The distal tube 26 includes a guide wire lumen 27 into which a guide wire may be inserted.

The operation unit 40 includes an operation unit main body 41 that contains the suction unit 80, the drive unit 70, the mixing unit 60, and a part of the discharge passage 50, and a connection portion 42 that is fixed inside the operation unit main body 41 and to which the proximal portion of the outer tube 23 is fixed. The connection portion 42 is provided with a hollow portion 43 having an internal space through which the drive shaft 22 rotatably penetrates. The internal space of the hollow portion 43 is in communication with the proximal end opening 25 of the drive shaft 22. The proximal end opening 25 is, for example, a hole penetrating from the inner periphery to the outer periphery of the proximal portion of the drive shaft 22. A ring-shaped seal portion 44 that seals the internal space of the connection portion 42 is disposed at a proximal portion of the connection portion 42. The outer periphery of the seal portion 44 is in close contact with the inner periphery of the hollow portion 43, and the inner periphery of the seal portion 44 is in close contact with the outer periphery of the drive shaft 22 in a slidable manner. The proximal portion of the outer tube 23 is fixed to the inner periphery of the hollow portion 43 on the distal side. The outer periphery of the hollow portion 43 is provided with a port 45 through which the internal space and the discharge passage 50 communicate with each other.

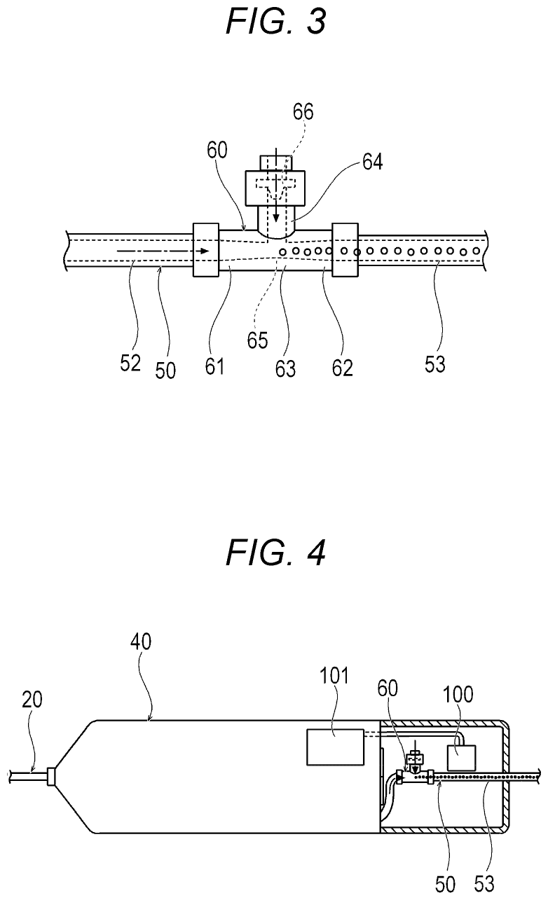

As illustrated in FIGS. 2 and 3, the discharge passage 50 includes a first passage 51 disposed between the port 45 and the pump 81, a second passage 52 disposed between the pump 81 and the mixing unit 60, and a third passage 53 disposed between the mixing unit 60 and the drainage bag 90. The first passage 51, the second passage 52, and the third passage 53 are formed of a tube. The first passage 51 can apply a suction force (negative pressure) of the pump 81 to the discharge lumen 21 via the port 45, the internal space of the hollow portion 43, and the proximal end opening 25 of the drive shaft 22. The second passage 52 can convey waste liquid discharged from the pump 81 to the mixing unit 60. The third passage 53 can convey the waste liquid having passed through the mixing unit 60 to the drainage bag 90. At least the third passage 53 of the discharge passage 50 is transparent or translucent so that an operator is enabled to visually observe the inner flow. The inner diameter of the third passage 53 is preferably larger than the inner diameter of the second passage 52 so that the fluid is easily mixed between the second passage 52 and the third passage 53. As an example, the inner diameter of the first passage 51 is 2.0 mm, the inner diameter of the second passage 52 is 2.0 mm, and the inner diameter of the third passage 53 is 2.2 mm.

The mixing unit 60 is a portion that mixes air into the discharge passage 50. The mixing unit 60 includes an inflow portion 61 coupled to a proximal portion of the second passage 52, an outflow portion 62 coupled to a distal portion of the third passage 53, a main flow path 63 disposed between the inflow portion 61 and the outflow portion 62, and a mixing flow path 64 in communication with the main flow path 63. The mixing unit 60 is disposed on a more downstream side than the suction unit 80. Therefore, it becomes possible to suppress a back-flow of the air, which has been mixed into the discharge passage 50 from the mixing unit 60, toward the upstream side beyond the suction unit 80. The mixing unit 60 may be disposed on a more upstream side than the suction unit 80.

The main flow path 63 includes a throttle portion 65 whose inner diameter decreases between the inflow portion 61 and the outflow portion 62. The mixing flow path 64 is in communication with the main flow path 63 substantially perpendicularly at the throttle portion 65. As an example, the inner diameter of the inflow portion 61 is 3.7 mm, the inner diameter of the outflow portion 62 is 3.7 mm, and the inner diameter of the throttle portion 65 is 1.6 mm. The main flow path 63 may not include the throttle portion 65 whose inner diameter decreases. For example, the main flow path 63 may have a constant inner diameter.

The mixing flow path 64 includes a check valve 66. The check valve 66 suppresses a flow from the mixing flow path 64 toward the outside while allowing a flow from the outside toward the mixing flow path 64. The check valve 66 is not particularly limited, and is, for example, a duckbill valve. The check valve 66 may be, for example, a membrane that allows gas to pass but does not allow liquid to pass. Furthermore, the mixing flow path 64 may have only an opening without the check valve 66.

Next, a method of using the medical device 10 according to the present embodiment will be described using, as an example, a case in which an area such as a thrombus or a calcified lesion in a blood vessel is a site that is to be destroyed and sucked.

First, the operator inserts a guide wire (not illustrated) into a blood vessel, and causes it to reach the vicinity of the area (e.g., thrombus or calcified lesion). Next, the operator inserts the proximal end of the guide wire into the guide wire lumen 27 of the medical device 10. Then, the medical device 10 is caused to reach the vicinity of the area using the guide wire as a guide.

Next, the operator actuates the drive unit 70 and the suction unit 80 of the medical device 10. As a result, rotation of the rotary drive shaft 71 and suction of the pump 81 start. The rotary drive shaft 71 rotates the drive shaft 22 and the cutting unit 30. The rotating cutting unit 30 cuts the area or site (e.g., thrombus or calcified lesion) inside the blood vessel.

The operating pump 81 applies a suction force (negative pressure) to the internal space of the hollow portion 43 via the port 45. Accordingly, a negative pressure is applied to the discharge lumen 21 of the drive shaft 22 from the proximal end opening 25 located in the internal space of the hollow portion 43. The area cut by the blade 31 of the cutting unit 30 becomes debris, and this debris is sucked into the discharge lumen 21 from the distal end opening 24 of the drive shaft 22.

The sucked drainage including the debris reaches the internal space of the hollow portion 43 from the proximal end opening 25 of the discharge lumen 21. The drainage in the internal space of the hollow portion 43 flows into the first passage 51 from the port 45, and is discharged to the second passage 52 through the pump 81. The drainage moved to the second passage 52 reaches the mixing unit 60.

The drainage reached the mixing unit 60 flows into the outflow portion 62 from the inflow portion 61 through the throttle portion 65. At this time, the drainage increases the flow velocity and decreases the pressure in the throttle portion 65 due to the Venturi effect. As a result, air (fluid) inside the operation unit main body 41 passes through the check valve 66, enters the main flow path 63 from the mixing flow path 64, and is mixed into or with the drainage. Since the check valve 66 is provided, leakage of the drainage from the mixing flow path 64 to the inside of the operation unit main body 41 is suppressed.

The flow velocity inside the tube is slower near the internal surface of the tube, and is faster as going farther from the internal surface of the tube. Thus, the pressure inside the tube is lower near the internal surface of the tube. Therefore, even if the throttle portion 65 is not provided in the main flow path 63, air (fluid) enters the main flow path 63 from the mixing flow path 64, and is mixed into the drainage.

Since the air that flows into the main flow path 63 from the mixing flow path 64 intermittently flows without being an uninterrupted steady flow, the air may be foam-like (granular) with air bubbles. The air that flows into the main flow path 63 from the mixing flow path 64 may be foam-like due to a turbulent flow even in the case of the uninterrupted steady flow. In particular, since the pump 81 intermittently sends the drainage, the air that flows into the main flow path 63 from the mixing flow path 64 also tends to be intermittent, and tends to be foam-like (granular). Moreover, when the check valve 66 has a structure that causes delay in mechanical movement like a duckbill valve, air (fluid) that passes through the check valve 66 tends to flow intermittently. Therefore, the drainage and the air are easily distinguished by contrast through visual observation. Accordingly, the operator is enabled to easily determine how much air is mixed in the drainage passing through the third passage 53 by visual observation. No air is mixed into the drainage from the mixing flow path 64 when no drainage is flowing. Therefore, the operator may easily determine whether or not drainage is flowing by determining whether or not air is mixed in the drainage.

The temperature of the air inside the operation unit main body 41 is easily raised by the first motor 72 and the second motor 82 to be driven. When the temperature of the air inside the operation unit main body 41 is excessively raised, the operation of the drive unit 70 and the suction unit 80 of the medical device 10 may become unstable. However, the air inside the operation unit main body 41 is discharged from the mixing unit 60 through the discharge passage 50. When the air inside the operation unit main body 41 is discharged, outside air flows into the operation unit main body 41 from a gap or the like of the operation unit main body 41. As a result, the inside of the operation unit main body 41 is cooled so that the operation of the drive unit 70 and the suction unit 80 of the medical device 10 may be stabilized.

The drainage and the air sent to the third passage 53 flow through the third passage 53, and are discharged to the drainage bag 90. After the cutting of the area (e.g., thrombus or calcified lesion) and the suction of the debris (cut debris) are complete, the operator stops the operation of the medical device 10. Accordingly, the rotation of the drive shaft 22 stops, and the suction of the pump 81 also stops. As a result, the cutting by the cutting unit 30 and the discharge of the debris stop. Then, the medical device 10 is removed from the blood vessel, and the procedure is complete.

As described above, the medical device 10 according to the present embodiment is the medical device 10 that discharges an object from a biological body lumen, and includes the shaft 20 that extends from the proximal side toward the distal side and includes the discharge lumen 21 through which the object can pass to the proximal side, the discharge passage 50 in communication with the proximal portion of the discharge lumen 21, and the mixing unit 60 capable of supplying fluid (e.g., air) to the discharge passage 50.

The medical device 10 configured as described above is capable of mixing, using the mixing unit 60, fluid into the flow toward the proximal side through the discharge lumen 21. Accordingly, the medical device 10 is enabled to improve discriminability of presence or absence of a discharging flow by creating appearance contrast in the flow. That is, the medical device is able to better discriminate or distinguish between the presence of a discharging flow and the absence of a discharging flow by creating a contrasting appearance in the flow. Furthermore, the fluid mixed by the mixing unit 60 does not flow toward the upstream side, whereby the fluid is not inadvertently mixed into the biological body lumen. Therefore, the present medical device 10 is highly secure.

Furthermore, at least a part of the discharge passage 50 on a more discharge side than the mixing unit 60 is transparent or translucent. Thus, the operator is enabled to easily recognize presence or absence of a discharging flow by visual observation.

Furthermore, the medical device 10 includes the pump 81 that is coupled to the discharge passage 50 and sends fluid toward the discharge side. Thus, it becomes possible to effectively apply the suction force to the discharge lumen 21.

Furthermore, the mixing unit 60 is disposed on the discharge side of the pump 81. Thus, it becomes possible to suppress a back-flow of the fluid, which has been mixed into the discharge passage 50 from the mixing unit 60, toward the upstream side beyond the pump 81.

Furthermore, the medical device 10 includes the operation unit 40 that is coupled to the proximal portion of the tube and contains the pump 81, and the mixing unit 60 is disposed inside the operation unit 40. Thus, it becomes possible to discharge, using the mixing unit 60, the air heated by the heat generated by the pump 81 from the discharge passage 50. Therefore, it becomes possible to cool the inside of the operation unit 40 to stabilize the operation of the medical device 10. With the mixing unit 60 disposed near the first motor 72 and the second motor 82 that easily generate heat, it becomes possible to effectively cool the first motor 72 and the second motor 82.

Furthermore, the pump 81 has a structure that intermittently reduces the pressure inside the discharge lumen 21 and sends the fluid. Thus, the fluid mixed in the flow passing through the discharge passage 50 tends to be intermittent, whereby discriminability of presence or absence of a flow may be improved.

Furthermore, the mixing unit 60 includes the main flow path 63 that communicates with the discharge passage 50 and is provided with, in a part of the flow direction, the throttle portion 65 having a reduced inner diameter, and the mixing flow path 64 that communicates with the main flow path 63 at the throttle portion 65, and the main flow path 63 and the mixing flow path 64 form a Venturi tube. Thus, it becomes possible to easily mix, from the mixing unit 60, fluid or a moving body into the throttle portion 65 in which internal pressure decreases due to the Venturi effect. Since the Venturi effect is caused by fluid flowing through the discharge passage 50 and is not caused when there is no flow, presence or absence of a flow may be easily recognized.

Furthermore, the mixing unit 60 includes the check valve 66. Accordingly, it becomes possible to suppress unintentional discharge of the flow, which flows toward the proximal side through the discharge lumen 21, from the mixing unit 60.

Furthermore, the pressure reduced by the pump 81 may open the check valve 66. The moving body may be air.

The present invention is not limited to the embodiment described above, and various modifications may be made by those skilled in the art . For example, the biological body lumen into which the medical device 10 is inserted is not limited to a blood vessel, and may be, for example, a vessel, a ureter, a bile duct, a fallopian tube, a hepatic duct, or the like. Furthermore, the medical device 10 may not include the cutting unit 30 and the drive shaft 22. In this case, the discharge lumen 21 is provided inside the shaft 20, which is a tube.

Furthermore, as in a variation illustrated in FIG. 4, the medical device 10 may include a bubble sensor 100 and a display unit 101. The bubble sensor 100 is disposed inside the operation unit 40, for example, and is capable of counting the number of bubbles flowing through the third passage 53 toward the drainage bag 90. The bubble sensor 100 is, for example, an ultrasonic sensor, and a structure thereof is not limited as long as bubbles can be detected. The display unit 101 calculates a flow velocity from an output result of the bubble sensor 100, and displays it. The display unit 101 includes, for example, a monitor and an arithmetic unit such as a central processing unit (CPU).

Furthermore, what is mixed into the discharge passage 50 from the mixing unit 60 may be a gas other than air, a colored (e.g., white) liquid, or a moving body that is a solid movable in the discharge passage 50. In the case of a liquid, the liquid is preferably a high-viscosity fluid so as not to be mixed with the waste liquid flowing through the discharge passage 50 to form a steady flow. The high-viscosity fluid is a fluid having viscosity higher than that of a body fluid (e.g., blood) included in the drainage, and is, for example, a contrast medium or a hypertonic solution. The fluid is not limited to a high-viscosity fluid, and may be, for example, a physiological salt solution. Furthermore, the fluid may be an oil-based fluid hardly mixed with a body fluid (e.g., blood). The moving body is, for example, a sphere (bead) having a size capable of moving in the discharge passage 50. In a case where a material mixed into the discharge passage 50 from the mixing unit 60 is other than air, a supply tube or a container for supplying a fluid or a moving body to be mixed is coupled to the mixing flow path 64.

Furthermore, the suction force may be generated not by the pump 81 but by the drive shaft 22. The drive shaft 22 may generate the suction force by rotating as long as it has a structure of an Archimedean screw pump. In this case, the medical device 10 may discharge debris without being provided with the pump 81.

Furthermore, a vacuum container sealed in a state where internal pressure is lower than atmospheric pressure may be used as the drainage bag 90, whereby the suction force may be generated by the vacuum container. In this case, the medical device 10 is enabled to discharge debris using the suction force of the vacuum container without being provided with a pump.

The detailed description above describes embodiments of a medical device representing examples of the new medical device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

10 medical device
20 shaft
21 discharge lumen
22 drive shaft
30 cutting unit
40 operation unit
50 discharge passage
60 mixing unit
63 main flow path
64 mixing flow path
65 throttle portion
66 check valve
70 drive unit
80 suction unit
81 pump

What is claimed is:

1. A medical device that discharges an object from a biological body lumen, the medical device comprising:
a shaft that includes a rotatable part having a proximal portion and a distal portion, the shaft including a discharge lumen having an open distal end, the discharge lumen extending from the open distal end to a proximal portion of the discharge lumen;
a cutting unit fixed to the distal portion of the rotatable part;
a motor connected to the rotatable part to rotate the rotatable part and the cutting part so that during use of the medical device when the motor is operated, the cutting unit is rotated to cut the object;
a suction producer in fluid communication with the discharge lumen so that operation of the suction producer during use of the medical device produces suction in the discharge lumen that causes the object cut by the cutting unit to enter the discharge lumen by way of the open distal end;
a discharge passage that communicates with the proximal portion of the discharge lumen; and
a mixing unit that supplies fluid or a moving body to the discharge passage during use of the medical device.

2. The medical device according to claim 1, wherein the discharge passage includes a first passage, a second passage and a third passage, the first passage having a distal end connected to the proximal portion of the lumen and having an opposite proximal end, the second passage having a distal end downstream of the proximal end of the first passage with respect to a direction of flow of the object and having an opposite proximal end connected to the mixing unit, the third passage having a distal end connected to the mixing unit and having an opposite proximal end, the distal end of the third passage being downstream of the proximal end of the second passage so that the mixing unit is positioned between the proximal end of the second passage and the distal end of the third passage.

3. The medical device according to claim 2, wherein the mixing unit includes a main flow path that extends between an inflow portion of the mixing unit and an outflow portion of the mixing unit, the proximal end of the second passage being connected to the inflow portion of the mixing unit, the distal end of the third passage being connected to the outflow portion of the mixing unit.

4. The medical device according to claim 3, wherein the mixing unit includes a mixing flow path that intersects and is in communication with the main flow path at a location along the main flow path that is intermediate the inflow portion and the outflow portion.

5. The medical device according to claim 4, wherein the mixing flow path of the mixing unit includes a check valve.

6. The medical device according to claim 4, wherein the main flow path and the mixing flow path forming a Venturi tube.

7. The medical device according to claim 2, wherein the suction producer includes a pump, the pump being in fluid communication with the second passage to pump the object toward the proximal end of the second passage.

8. The medical device according to claim 7, wherein the pump and the mixing unit are both housed inside an operation unit that is connected to a proximal portion of the shaft, the second passage also being located inside the operation unit, and operation of the pump creating the suction inside the operation unit so that the suction acts on the proximal portion of the discharge lumen to object cut by the cutting unit to enter the discharge lumen by way of the open distal end to cause the object cut by the cutting unit to enter the discharge lumen by way of the open distal end.

9. A medical device that discharges an object from a biological body lumen, the medical device comprising:
a shaft that includes a discharge lumen opening to a distal portion of the shaft and extending to a proximal portion of the shaft;
a discharge passage that communicates with the discharge lumen;
a mixing unit that supplies fluid or a moving body to the discharge passage during use of the medical device;
the mixing unit and the discharge passage being enclosed within an enclosure of an operation unit, and a proximal portion of the shaft being located in the enclosure of the operation unit;
the mixing unit that is enclosed within the enclosure of the operation unit comprising a main flow path positioned intermediate opposite ends of the discharge passage, the main flow path having an inflow portion and an outflow portion that are spaced apart from one another, the mixing unit that is enclosed within the enclosure of the operation unit also comprising a mixing flow path that intersects and is in fluid communication with the main flow path at a location along the main flow path that is intermediate the inflow portion and the outflow portion, the mixing unit that is enclosed within the enclosure of the operation unit also comprising a one-way valve allowing the fluid or the moving body to be introduced into the discharge passage by way of the main flow path.

10. The medical device according to claim 9, further comprising a pump that is operable to create suction that acts on the discharge lumen in the shaft to drawing the object into the discharge lumen by way of an opening at a distal end portion of the shaft.

11. A medical device that discharges an object from a biological body lumen, the medical device comprising:
a shaft that extends from a proximal side toward a distal side and includes a discharge lumen through which the object can pass to the proximal side;
a discharge passage that communicates with a proximal portion of the discharge lumen; and
a mixing unit that supplies, to the discharge passage during operation of the medical device, fluid or a moving body movable in the discharge passage.

12. The medical device according to claim 11, wherein at least a part of the discharge passage that is located proximal of the mixing unit is transparent or translucent.

13. The medical device according to claim 11, further comprising: a pump coupled to the discharge passage and operable to send the object and bodily fluid toward a discharge side together with the fluid or moving body supplied at the mixing unit.

14. The medical device according to claim 13, wherein the mixing unit is disposed on the discharge side of the pump.

15. The medical device according to claim 13, further comprising:
an operation unit that is coupled to a proximal portion of the shaft and contains the pump, wherein
the mixing unit is disposed inside the operation unit.

16. The medical device according to claim 13, wherein the pump has a structure that intermittently reduces pressure inside the discharge lumen and sends the fluid toward the discharge side.

17. The medical device according to claim 16, wherein the mixing unit includes a check valve.

18. The medical device according to claim 17, wherein the check valve is openable in response to the pressure reduced by the pump.

19. The medical device according to claim 11, wherein the mixing unit includes a main flow path that communicates with the discharge passage and includes a throttle portion having a reduced inner diameter, and a mixing flow path that communicates with the main flow path at the throttle portion, and the main flow path and the mixing flow path forming a Venturi tube.

20. The medical device according to claim 11, wherein the moving body includes air.
